(19) United States
(12) Patent Application Publication
(10) Pub. No.: US 2019/0018012 A1
(43) Pub. Date: Jan. 17, 2019

(54) Method and System for Addressing the Placebo Effect to Ameliorate its Practical Effect on Drug Testing

(71) Applicant: Joseph E. Kovarik, Englewood, CO (US)

(72) Inventor: Joseph E. Kovarik, Englewood, CO (US)

(21) Appl. No.: 16/037,053

(22) Filed: Jul. 17, 2018

Related U.S. Application Data
(60) Provisional application No. 62/533,358, filed on Jul. 17, 2017.

(51) Int. Cl. G01N 33/569

(52) U.S. Cl. CPC G01N 33/56911

(57) ABSTRACT

A system and method for providing improved therapeutic treatments and improved clinical trials for therapeutic treatments involves the predicting of a placebo response or effect by appreciation of the condition of and variances to the microbiome of individuals involved in drug efficacy studies. The provision of purportedly neutral placebos, such a “sugar pill”, has an effect on the microbes in a person's body, and therefore often has an effect on the person's wellbeing and perception of treatment. An appreciation that there is an actual effect due to the modification of the microbiome, caused by the administration of the placebo, forms a basis for various methods and systems of the present invention, with such appreciation being employed to design and conduct better drug efficacy studies without the prior problems experienced due to the placebo effect.

### RELATED APPLICATIONS

This application is a non-provisional of U.S. Provisional Patent Application Ser. No. 62/533,358 filed on Jul. 17, 2017. The entire disclosure of the prior application is considered to be part of the disclosure of the accompanying application and is hereby incorporated by reference.

### FIELD OF THE INVENTION

Certain aspects of the present invention pertain to the technical field of methods for providing improved therapeutic treatments and improved clinical trials for therapeutic treatments. More particularly the present invention relates to methods/systems for predicting a placebo response or effect and to systems providing such predictions and using the generated data of the predictions. Various embodiments of the present invention are directed to the cause of the placebo effect being due to variances of the microbiome of a person and the prior failure to appreciate that provision of purportedly neutral placebos, such a “sugar pill”, feeds certain microbes in a person's body and therefore has an effect on the person's wellbeing and perception of treatment—due to there being an actual effect caused by the modification of the microbiome as a result of the administration of the placebo.

### BACKGROUND OF THE INVENTION

A placebo is defined as a medically inert substance or technique, which is administered like a drug. As the staple negative control in clinical trials, placebos play a critical role in modern medicine. The placebo effect refers to the well-documented phenomenon in which patients feel better after receiving a placebo and in many situations, there is an actual biological result despite being provided with a purportedly neutral agent.

Individuals receiving placebo treatments often experience an improvement in their condition, or alternatively, can experience side effects similar to those exhibited by individuals receiving drug treatment. Such a reaction to an “inert” treatment i.e. a treatment lacking an active ingredient is termed the “placebo effect”. A potential therapeutic must be assessed and approved for clinical efficacy and safety before it can be marketed. In cases where a drug entity is being evaluated for treatment of a condition where there is no conventional therapy available for treatment, the new drug is generally compared with a placebo. A placebo is a mixture lacking any active ingredients. Clinical trials are structured to comprise a drug treatment group and a placebo treatment group. Typically, the clinical investigators and the subjects of the trial are blinded to the assignment of subjects in these groups. The pharmaceutical industry spends billions of dollars every year in preclinical and clinical research in its quest for new therapeutics. It would be a significant benefit to the pharmaceutical industry if one could simply avoid or reduce the impact of the placebo effect on the data derived from a clinical trial.

Predicting who will be a placebo responder would be of great value to researchers and patients. In drug development, detecting a difference between active intervention and the placebo control is an underlying goal of randomized controlled trials (RCTs). Being able to identify and exclude individuals who are more likely to respond to placebos enables researchers to enhance trial designs seeking to find such a difference. Potential cost savings due to reduction of sample size would be of enormous benefit for drug development.

The clinical development of new drugs or treatments in major therapeutic indications is complex and is not efficient. Many Phase 2 and 3 clinical trials are abandoned or fail because of the inability to demonstrate clear superiority of the tested drug versus a placebo despite promising results observed in vitro and/or in pre-clinical studies. The reason for this is that, in therapeutic fields, the placebo response by itself has been shown to have had a pronounced effect on the primary outcomes of the clinical studies. Properly managing the placebo effect/response may positively contribute to the better well-being of patients and is also essential to design appropriate clinical trials to allow a clear differentiation between the effect of the studied drug and the other effects collectively referred to as the placebo effect. The placebo response on the drug efficacy evaluation and the absence of common traits among patients that allow one to measure to what extent the placebo response interferes with the assessment of a new drug candidate, make it very difficult to demonstrate a new drug's superiority. As a result, both clinical research scientists and pharmaceutical companies need improved clinical study designs and improved patient's characterization so as to differentiate the placebo response from the effect of the tested drug.

The cause for the placebo effect has been a long felt but unsolved problem. Understanding the placebo effect could help in filtering out noise in clinical trials and in avoiding flawed study designs, and ultimately, in providing the public with useful treatments to various disease that would otherwise not be pursued due to the placebo effect.

### SUMMARY OF THE INVENTION

For drug companies, the placebo effect is a nemesis. The placebo effect is actually many different effects that vary based on the symptom or disease in question. While some contend that this phenomenon encompasses many contextual factors surrounding a clinical encounter, including patient beliefs and expectations, practitioner attitude, clinic environment, patient-practitioner communication, and method of treatment administration (whether placebo or active), the present inventor submits that the majority of the effects observed are due to the variety and changes that occur to an individual's microbiome when the comparative tests of a placebo vs. an active ingredient are tested. Thus, while there may be certain psychological factors involved in any treatment result, the majority of issues encountered when performing tests lies in the failure to appreciate that the differences of a person's microbiome and the effect of a seemingly innocuous sugar or starch pill on such microbiomes, can have profound effects on an individual's microbiome, and thus, the results obtained in comparing such purportedly neutral compositions with active ingredients becomes skewed. To address such a concern, first one must appreciate the fact that the microbiome plays a role in most every biological experiment and thus, factoring the implications of the differences and effects on an individual's microbiome is one way to assure that the comparative results are more meaningful and accurate. Ascribing some mystical or unknowable cause to the placebo effect has done far more damage than good in having valuable and meaningful medical interventions made available to patients suffering from a variety of ailments.

The present invention provides methods and systems for elucidating the underlying biological bases for placebo effects and provides a way to design clinical trials in drug screening and the like to limit the placebo effect, and thus to provide more accurate information and results of drug tests to determine which may be truly effective. From a clinical perspective, knowing likely responders provides a way to effectively modify treatment approaches to allow for more careful titrations of medication dosages. Precise knowledge of the contribution of an individual's microbiome and the variation of the microbiomes of those in the testing group as it relates to placebo effects is one way in which to guide the development of more efficient controls in experiments and refinements of clinical practice.

The recent availability and appreciation of the many ways humans are affected by the microbiome offers a way to approach and understand, control, and harness the placebo response. Almost half the cells and 1% of the unique genes found in our bodies are human, the rest are from microbes, predominantly bacteria, archaea, fungi, and viruses. These microorganisms collectively form the human microbiota, with most colonizing the gut.

It is difficult to define a healthy microbiome of a person as there is so much variability involved. It is more productive to adopt a paradigm to explain ways to establish complex causation by pursuing an ecological approach that recognizes that disease affects not the host, but the holobiont in all its complexity. Thus, when testing drugs and employing a placebo, the effect on the diversity of microbiota presents an intricate system composed of microbial individuals interacting in a social fashion. Consequently, when trying to understand holobionts in health and disease, parameters such as microbial diversity of the microbiota, predominance or absence of specific species of bacteria, levels of defined metabolites, etc. may be employed to define the baselines for comparative analysis.

The composition of the gut microbiota is in constant flow under the influence of factors such as the diet, ingested drugs, the intestinal mucosa, the immune system, and the microbiota itself. Within the human digestive tract, there are trillions of bacteria, and these communities contain hundreds or even thousands of species. The makeup of those populations can vary greatly from one person to another, depending on factors such as diet, environmental exposure, and health history. The human gut microbiome maintains a relatively stable state within individuals despite its exceptionally high species diversity. Diversity per se, however, does not necessarily equate to a stable microbiome since a large number of interacting species tends to have a destabilizing effect. For example, some studies have found that the gut microbiome of formula-fed infants is more diverse but less stable compared to breast-fed infants. Nevertheless, other numerous studies suggest that reduced microbiome diversity may be associated with ill health.

Accordingly, certain aspects of various embodiments of the present invention are directed to a system and method of identifying a biological basis for a placebo effect. A placebo may comprise one or more inert ingredients, or even one or more active ingredients (e.g., where the activity is expected to be orthogonal to the therapy at issue). In many cases, sugar pills or starch pills are employed as a placebo to test an experimental drug. The effect of such presumably innocuous elements as sugar or starch have traditionally been considered to be worthy of no notable importance. What the present inventor contends, however, is that these admittedly simple constituents have a profound effect on various biological systems due to the involvement of an individual's microbiome. It is the past failure to appreciate the variety of any given individual's microbiome's and the effect of so-called placebos on such microbiomes that accounts for the seemingly mysterious beneficial effects of such simple constituents being administered to such individuals, as compared to those who are administered active new drug components.

Thus, in certain embodiments of the present invention, biological markers are screened for correlation to the placebo effect by providing such placebo ingredients to an individual prior to employing such individual in a drug testing scenario. In such a manner, one is able to determine whether the presumed inert component of the placebo actually does have a biological response due to the administration of the presumably inert component. It is believed that many individuals who are administered such presumably inert components will find that their microbiomes are affected by such components in a manner that will significantly and often substantially affect the results of testing for a particular biological event.

While not bound by theory, the present inventor contends that one reason the placebo effect has been under its long veil of mystery is that traditional experiments did not employ, in addition to a placebo, a no-treatment control (NTC). A NTC is one of the few methodologies that can disentangle genuine psychosocial and physiological placebo responses to the symbols, rituals, and behaviors of the clinical encounter from spontaneous remission, regression to the mean, and the natural waxing and waning of illness. Without studies that have NTCs, a control for the placebo arm, an accurate and comprehensive view of what is actually going on in clinical trials is difficult, if not impossible to achieve.

Although we do not yet have a comprehensive understanding of the microbiome, it is prudent to consider issues that arise and the potential impact on RCT design as the known variability of the microbiome amongst people varies, and thus, one would expect challenges with confounding, potential microbiome-drug-placebo effect modifications and disease specific effects. An important underlying assumption in RCTs is that, in aggregate, the main difference between the drug treatment and placebo arms is solely the effect of the active drug. One aspect of the present invention is directed to refining and re-calibrating the assumptions of placebo controls in RCTs so that the microbiome of individuals is taken into account, thus providing a method and system that provides a far better and more accurate assessment of the efficacy of active drugs being tested and compared to placebos.

A purported placebo may be analyzed for a presence or absence of one or more biological effects on an individual's microbiome prior to the testing of the individual using the new drug to be tested and compared against the biological evaluation as compared to the placebo. One objective of the present invention is to provide a more consistent ability to achieve replication of studies with proper placebo controls to take into account the impact of microbiome differences and thus, the differential placebo responses observed, and in such a manner, assist in determining an initial condition of individuals when taking part in a study such that microbiome conditions are acknowledged and accounted for. Personalizing drug doses based on microbiome factors and profiles is important and is significant in evaluation of diseases and conditions, such as pain, functional urinary and bowel conditions, symptoms of fatigue, nausea, hot flashes, depression, anxiety, irritable bowel syndrome, acute episodic migraine attack, and depression, is believed to enhance the confidence that researchers have in results from trials, making such tests far more meaningful and feasible with knowledge of a patients' microbiome.

The placebo response is a complex phenotype with an unfolding physiology. The microbiome of any individual consists of multiple intersecting pathways that have upstream or downstream effects on dopamine and opioid function, depending on the disease or disorder being treated. The overlap between placebo, drug treatment and disease add to the complexity of the microbiome and underscores the importance of understanding how it fits into larger more complex biological networks. Knowledge of the microbiome is employed in various embodiments of the present invention to guide the practice of strategies for identifying placebo responders and clinical trial design. Given the benefits in terms of research design, reduction in the cost of clinical trials, and safer more effective personalized medicines, the present invention in its various embodiments provides a solution to what has been a long sought but unsolved solution to the problems implicated by the mysterious “placebo effect.”

The efficacy of a drug is determined by the difference between the aggregate outcomes of individuals randomized to a drug versus placebo treatment. Thus, certain embodiments described herein provide a method of screening individuals for a placebo effect correlation with respect to the microbiome of such individual, prior to employing such individual to test for the effectiveness of a new drug on such individual, so as to address whatever significant changes in the biological responses such individual may have after being administered the purported neutral placebo co-promotion.

A correlation to a placebo effect can be useful in designing clinical trials by accounting for the placebo effect in the relevant clinical trial or experiment as it may be affected by the microbiome of the individual. Various embodiments of the present invention therefore provide for the design of clinical or other trials to account for the placebo effect, taking biological predispositions to the placebo effect into account. Thus, in various embodiments, a system and method is provided for testing a placebo-normalized activity of a putative therapeutic agent which for the first time, accounts for various microbiota-based factors of those involved in clinical trials that employ placebos.

Normalizing activity data optionally includes revising estimates of a correlation between the desired activity and administration of the putative therapeutic agent, based upon an expected placebo effect on this correlation. That is, the expected placebo effect normalization takes into account the effect on the population members that have microbiome predisposed to display the placebo effect. Such normalizing is optionally performed after obtaining a complete desired activity data set corresponding to the desired activity of the therapeutic agent in the first population, or can be performed during a process of obtaining a desired activity data set corresponding to the desired activity of the therapeutic agent in the first population.

Certain aspects of the present invention are directed to systems and methods for screening an individual or population for susceptibility to a placebo effect, with such screening being focused on the effect an individual's microbiome may have on the purported placebo. By taking the microbiome into account, the veracity and meaning of results from clinical trials is advanced exponentially and the mystery of previous placebo effects is minimized. In other words, the confounding and confusing results observed that have been attributed to the placebo effect can be viewed as having a biological basis that depends from the nature of the microbiome's of individual's involved in such studies.

While there are a multitude of variations possible in view of this recognition of what the true nature of the placebo effect problem consists of, certain embodiments of the present invention involve the acknowledgment of the microbiome on the placebo effect and the neutralizing of such variations in a multitude of ways, including but not limited to pre-testing individuals for the effects they experience after being administered a placebo to determine how the individual microbiome's of such individual may be affected thereby. Assessment of the microbiome of individuals included within RCT's also forms a step in method of certain embodiments of the present invention.

One will appreciate that this Summary of the Invention is not intended to be all encompassing and that the scope of the invention nor its various embodiments, let alone the most important ones, are necessarily encompassed by the above description. One of skill in the art will appreciate that the entire disclosure, as well as the incorporated references, pictures, etc. will provide a basis for the scope of the present invention as it may be claimed now and in future applications. While embodiments of the invention have been illustrated and described, it is not intended that these embodiments illustrate and describe all possible forms of the invention. Rather, the words used in this specification are words of description rather than limitation, and it is understood that various changes may be made without departing from the spirit and scope of the invention.

### DETAILED DESCRIPTION OF VARIOUS EMBODIMENTS OF THE PRESENT INVENTION

In order to be approved by the Federal Drug Administration (FDA), new drugs must prove more efficacious than placebos in clinical trials. If a drug is unable to outperform a placebo in a clinical trial, it's fair to conclude that the drug isn't good enough to receive approval. In the pain field, distinguishing between placebo and drug effects can be tricky because both appear to activate the same mechanisms, such as the release of endorphins, which can mask drug effects in painkiller trials.

The placebo effect has traditionally been regarded as something negative, as a seemingly unexplained mystery at the heart of modern medicine. However, as we are beginning to understand the biological mechanisms underlying the placebo effect, it is becoming increasingly evident that placebos present a unique opportunity to understand the effect of the microbiome of individuals and how microbes can affect the results of RCT's.

In the 1980's, neuroscientist Jon Levine conducted what is now considered one of the quintessential analyses of the placebo effect. Postoperative patients received either a secret dose of 6-8 mg of morphine, or an overt dose of a substance described as a powerful painkiller (but was actually saline solution). The results were remarkable: patients in both groups reported the same degree of pain relief.

Prior studies of individuals with irritable bowel syndrome showed that the placebo effect worked almost as well as the leading medication on the market. This was found to be true even when the individuals were informed that the medication they were taking was a “fake” otherwise known as a placebo. Researchers were surprised that the placebo seemed to work for many of the individuals even though the individuals were aware that the medication was a placebo. In fact, according to the study, it was made absolutely clear to the individuals that the placebo medication contained no active ingredient and was made from inert substances and the term “placebo” was actually printed on the bottle. The individuals were even told not to believe in the placebo effect and to just ingest the medication.

Certain aspects of the present invention include the appreciation that there exists a tremendous variety of differences between individual microbiomes and that such differences account for the results observed in many placebo studies. The fact is that placebos very often elicit biological responses. The involvement of an individual's microbiomes on RCT's results and in particular, how placebos affect an individual's microbiome in a manner that can skew experimental results where the researcher has (wrongly) assumed that the placebo administered would have little, if any effect, is one aspect of various embodiments of the present invention. Thus, contrary to popular belief, patients don't just imagine placebo responses. Rather, numerous brain-imaging studies have confirmed that placebos cause measurable changes in neurobiological signaling pathways. The present inventor contends that a mere sugar pill may lead to a tangible physical response due to the impact such alleged placebo has on the microbiome of the individual. While placebos are sometimes grounded in a patient's psychological reaction to receiving treatment, placebos, due to their impact on an individual's microbiome, also act via the same biological pathways as painkillers.

One aspect of the present invention is directed to a method for predicting a placebo response in an individual, comprising testing the purported placebo on an individual prior to an actual test being conducted using a subject new drug so as to assess the effect of the placebo on the microbiome of the individual. Attributing a Scoring Factor to said individual, whereby said Scoring Factor is a measure of propensity to raise a placebo response and/or a measure of the intensity of said response, can be employed to as to address what otherwise may be skewed results form a typical placebo controlled experiment.

Certain preferred embodiments are directed to a method for predicting a placebo response in an individual, comprising a first administration of a purported placebo to an individual prior to an actual testing of a new drug in a placebo controlled trial, with data obtained with respect to the effect of the purported placebo compound/composition on the microbiome of the individual. The microbiome effect is then noted and employed when such individual is used in the actual trial for the new drug, such that the effects of the placebo component may be more accurately assessed, as compared to traditional trial where it was merely assumed that the placebo would have no effect whatsoever.

Such data may be used in a mathematical model stored on a computer for computing a correlation between the input data, thereby attributing a Scoring Factor to said individual, whereby said Scoring Factor is a measure of propensity to raise a placebo response and/or a measure of the intensity of said response. The Scoring Factor may be compared to a cut-off value to determine a classification whether or not a placebo response exists in an individual.

The placebo effect is multifactorial in nature and each individual may demonstrate a different response based on his/her therapeutic history and personality related aspects. It has also been observed that the placebo-effect is disease dependent, whereby an individual will show an effect which differs from disease to disease. It has also been reported that the placebo-effect is time dependent, whereby an individual will show a placebo response which evolves with time or time of treatment. Hence patients may respond to a placebo effect differently at the start of a treatment compared to the level of response during or at the end of a treatment. All of these acknowledgments are consistent with the placebo effect having a root cause in the changing nature of the microbiome of individuals. As such, various aspects of the present invention are directed to acknowledging such microbiota conditions and employing such knowledge in designing drug trials such that far more accurate reporting of a new drug's effectiveness can be assessed. Hitherto, the microbiome of the individual was not considered, and hence, the seemingly mysterious results from drug trials, here the placebo seems in various scenarios, to be nearly as effective as the new drug.

There are several mechanisms that are strongly implicated in the placebo response, all of which can be influenced by an individual's particular structure and composition of their microbiome: including activation of endogenous opioid and cannabinoid systems, dopamine release; changes in brain activity, and modulation of immune function or hormone release. In many cases, these effects mirror the effect that an active drug might have when treating the same condition. For example, placebos for treating pain often trigger the release of endogenous opioids, which have the same effect as pain-relieving drugs like morphine that supply external opiate agonists.

Even trials conducted employing a “no treatment” group fail to take into account the variety and diversity of individuals' microbiomes and thus, test results are skewed, even without a simple administration saline or sugar pill. Some of the diseases that have exhibited large placebo responses can now been seen as being those diseases where the microbiome is implicated. For example, Parkinson's disease patients have historically exhibited large placebo responses in clinical trials, with placebos triggering dopamine release, which directly alleviates many of the symptoms of Parkinson's. The recent appreciation of the microbiome in Parkinson's disease is believed to be an explanatory factor and provides supporting evidence of the placebo effect observed in comparative treatment trials of such disease. Similarly, placebo effects also appear to be clinically relevant for depression and anxiety, both of such conditions also now known to be affected by the structure and composition of one's microbiome. The involvement of an individual's microbiome may also assist in explaining previously confounding findings of participants in a placebo group of clinical trials—who receive no drugs—nevertheless reporting supposedly drug-induced side effects. Similarly, a review of migraine medication trials also found high rates of adverse events reported among placebo groups, with some of these “side effects” corresponding to the side effects that would be expected from the active drug. The association of an individual's microbiome and migraines is explored in a co-pending patent application by the same inventors here, which is incorporated herein in its entirety.

An active placebo is a treatment that does have a physiologic effect but is not indicated for the present condition. One of the more problematic examples of this is prescribing antibiotics for a viral infection. Given the importance of an individual's microbiome on various disease states, including viral related diseases, the taking of antibiotics and the effects on not only resident bacteria, but on the mitochondria of an individual, is something that must be reevaluated with the appreciation of an individual's personal microbiome and how such “active placebos” affect the same. The present inventor contends that placebo responses are legitimate biological reactions that must be rigorously characterized to facilitate efficient pharmaceutical development and optimal clinical care. To comply with written description and enablement requirements, the following references are incorporated herein in their entireties for their disclosures: U.S. Pat. No. 7,335,474 to Margus et al.; WO 2015169810 to Pereira; 2017/0053082 to Pereira et al.; 2014/0006042 to Keefe; U.S. Pat. Nos. 9,549,842 9,445,936; 2017/0100328 U.S. Pat. Nos. 8,701,671; 9,408,880; 9,585,920; 9,457,077; 9,730,967; 9,750,802; 2017/0106026; 2017/0100330; 2017/0100329; 2017/0106025; 2017/0119827 and 2017/0348359 to Kovarik; 2014/0006042 to Paulson; WO 2005027719 to Cox; WO 2013039574 to Pashko; 2017/0300647 to Goldberg; U.S. Pat. No. 6,687,685 to Sadeghi et al, U.S. Pat. No. 7,761,312 to Brown and 2015/0315651 to Winkler.

In one particular embodiment, all test subjects are provided with an identical mucosal adhesive strip which has a predetermined amount and type of bacteria thereon. For especially oral administered (and targeted) drugs, the use of such strips to alter the oral microbiome of the individual participating in a trial is employed to provide a more consistent level of microbiota environment of the different individual's in any given study. Therefore, with some assurances that approximately the same content of microbiota is provided to all test subjects, there is a higher likelihood that differences in the individual microbiomes (here, specifically directed to the oral microbiome) will not be the factor involved in assessing results from a comparison of a purported placebo and a new drug being tested. The objective is to attempt to largely neutralize the effect that differences in any given individual's microbiome may have when assessing whether a drug candidate has functional attributes over and above the placebo provided to individuals in the trial.

In other embodiments, the pre-measurements of an individual's microbiota is performed so as to gauge how similar such microbiota may be to other individuals. The assessment of the content and composition of an individual's particular microbiome is something that one of skill in the art can evaluate, especially in view of recent abilities to identify microbes that traditionally have been difficult to cultivate in the lab. Various references incorporated herein by this reference can be relied upon for such teachings. In preferred embodiments, tests are conducted on new drugs such that the individual's tested (e.g. given either the new drug or the placebo) will have basically the same microbiota constituents, and therefore, will similarly respond to either the placebo or the drug, thus facilitating a better assessment of the biological functionality of the new drug. The fact that certain purported placebos might unwittingly serve to enhance certain aspects of a person's microbiome in a manner that would skew experimental results, is therefore addressed in various ways in the different embodiments of the present invention, by leveling the degree to which microbiota effects are causative factors in reported results. In other embodiments, the appreciation that every individual has his/her own unique constitution of a microbiota is considered when administering a placebo. For example, a pre- and post-placebo administration can be performed with a purported placebo and the microbiome of the individual assessed, in order to determine whether there may be an unexpected biological response to the purported placebo due to the involvement of the individual's microbiome's response thereto. By measuring such effect, the drug trials can more accurately be assessed and any individual who had reacted to the placebo by marking a profound change to his/her microbiome, can be similarly assessed in view thereof when the actual drug testing data is reported.

While specific embodiments and applications of the present invention have been illustrated, and described, it is to be understood that the invention is not limited to the precise configuration and components disclosed herein. Various modifications, changes, and variations which will be apparent to those skilled in the art may be made in the arrangement, operation, and details of the methods and systems of the present invention disclosed herein without departing from the spirit and scope of the invention. Those skilled in the art will appreciate that the conception upon which this disclosure is based, may readily be utilized as a basis for designing of other structures, methods and systems for carrying out the several purposes of the present invention. It is important, therefore, that the claims be regarded as including any such equivalent construction insofar as they do not depart from the spirit and scope of the present invention.

What is claimed is:

1. A method for reducing the likelihood of a placebo effect when determining the activity of a putative therapeutic agent, comprising,
providing a putative therapeutic agent demonstrating positive clinical and/or in vitro results;
identifying a first individual who is a placebo responder by determining whether a purportedly neutral component administered to the individual has a biological effect;
assessing a microbial diversity of a microbiota of said first individual;
correlating the microbial diversity of the first individual to a placebo effect to identify the microbial basis for the placebo effect, wherein the placebo effect is selected from the group of effects consisting of: an improvement in a condition of the first individual in response to administration of the purportedly neutral component, and a deterioration in a condition of the first individual in response to administration of the purportedly neutral component;
identifying a second individual who is a placebo responder by determining whether the purportedly neutral component administered to the second individual has a microbial effect;
assessing a microbial diversity of a microbiota of a second individual;
correlating the microbial diversity of the second individual to a placebo effect to identify the microbial basis for the placebo effect, wherein the placebo effect is selected from the group of effects consisting of: an improvement in a condition of the second individual in response to administration of the purportedly neutral component, and a deterioration in a condition of the second individual in response to administration of the purportedly neutral component;
comparing the microbial diversity of the first individual to the microbial diversity of the second individual;
assessing differences in the first individual's microbial diversity and the second individual's microbial diversity; and
selecting the second individual for a study of the putative therapeutic agent only if the microbial diversity of the second individual is substantially similar to the microbial diversity of the first individual.

2. The method of claim 1, further comprising, assessing factors including at least two of the following: diet; ingested drugs; environmental exposure; health history;
intestinal mucosa, the immune system of the first and second individuals.

3. The method of claim 1, further comprising, providing a no-treatment control.

4. The method of claim 1, further comprising, accounting for microbiota based factors.

5. The method of claim 1, further comprising, neutralizing variations in at least the first individual's microbiome by pre-testing at least the first individual for effects they experience after being administered a placebo to determine how the individual microbiome's of the at least first individual is affected.

6. The method of claim 1, further comprising, comparing at least a first biological marker of the first individual to said first biological marker for the second individual, correlating the first biological marker to the placebo effect to identify the biological basis for the placebo effect.

7. The method of claim 1, wherein the placebo is selected from the group consisting of a suspension, a tablet, and an intravenous injection.
